(19) United States
(12) Patent Application Publication
(10) Pub. No.: US 2024/0050280 A1
(43) Pub. Date: Feb. 15, 2024

(54) SYSTEMS AND METHODS FOR MANAGING OPERATION OF WOUND DRESSINGS OR WOUND TREATMENT DEVICES

(71) Applicant: T.J.Smith and Nephew,Limited, Hull (GB)

(72) Inventors: Edward Yerbury Hartwell, Hull (GB); Felix Clarence Quintanar, Hull (GB); Neill John Rawson, Doncaster (GB)

(21) Appl. No.: 18/270,454

(22) PCT Filed: Jan. 7, 2022
(86) PCT No.: PCT/EP2022/050229

§ 371 Date: Jun. 29, 2023

(30) Foreign Application Priority Data
Jan. 15, 2021 (GB) 2100527.7

(51) Int. Cl. A61F 13/00; G16H 40/40; G16H 20/40

(52) U.S. Cl. CPC A61F 13/00051; G16H 40/40; G16H 20/40

(57) ABSTRACT

A wound treatment system can include control circuitry configured to provide a drive signal to a wound dressing. The wound dressing can be configured to perform wound treatment based upon the drive signal. Prior to performing the wound treatment, the control circuitry can be paired with the wound dressing. The wound dressing can be configured to function only when connected to particular control circuitry after pairing of the wound dressing and the control circuitry. Further, based on pairing the wound dressing and the control circuitry, a timer can be initialized such that the wound dressing can be configured to function prior to the timer reaching a lifetime threshold and the control circuitry can be configured to disable the wound dressing, via a fuse, upon the timer reaching the lifetime threshold.

### TECHNICAL FIELD

Embodiments described herein relate to apparatuses, systems, and methods for the monitoring or treatment of wounds.

### DESCRIPTION OF THE RELATED ART

Chronic wounds and/or open wounds that do not heal within a normal timeframe are a significant problem amongst certain patient populations, particularly older patients that may have a compromised vasculature. Treatment of such wounds via application of negative pressure wound therapy (NPWT) to the wound site is well known in the art and numerous therapies exist. For example, NPWT systems and dressings such as PICO, RENASYS-F, RENASYS-G, RENASYS-AB, RENASYS-F/AB are currently available from Smith & Nephew.

The use of therapeutic ultrasound for the treatment of bone is also well known in the art. For example, the EXOGEN Ultrasound Bone Healing device by Bioventus is FDA-approved for accelerating the healing of bone fractures. However, application of therapeutic ultrasound to wounded tissue outside of bone is not well-known in the art, nor is the application of therapeutic ultrasound in combination with NPWT. Application of ultrasound to wounded tissue or unwounded tissue presents a number of complications, such as the proper mode of delivery and ideal signal parameters for healing intact and/or wounded tissue. Consequently, a proper vehicle for the delivery of therapeutic ultrasound to wounds is currently unknown.

Many different types of dressings are known in the art for use in wound healing. These different types of wound dressings include many different types of materials and layers, for example, gauze, pads, foam pads or multi-layer wound dressings. However, use of such dressings in combination with therapeutic ultrasound is not well-understood in the art, particularly due to the difficulties in transmitting therapeutic ultrasound through various mediums into tissue. Additionally, the optimum signal parameters for wound healing or for treating other types of tissues is not well-known.

Therefore, improved methods and techniques for delivering ultrasound to wounds and other tissues are needed.

### SUMMARY

A wound therapy apparatus can include a treatment source. The treatment source can be configured to supply stimulation. The treatment source can be configured to be connected to a wound dressing covering a wound. The wound dressing can include one or more electronic components. The one or more electronic components can be configured to provide treatment to the wound in response to the stimulation received from the treatment source. The wound treatment apparatus can include control circuitry. The control circuitry can be configured to, in response to detecting an initial connection of the treatment source to the wound dressing, communicate pairing data to the wound dressing. The control circuitry can be configured to, in response to detecting an initial connection of the treatment source to the wound dressing, start a timer configured to monitor operational lifetime of the wound dressing. The control circuitry can be configured to, in response to detecting a subsequent connection of the treatment source to the wound dressing, receive identification data from the wound dressing. The control circuitry can be configured to, in response to detecting a subsequent connection of the treatment source to the wound dressing and in response to a determination that the identification data matches the pairing data and that the timer has not reached a lifetime threshold, cause the treatment source to supply the stimulation to the wound dressing. The control circuitry can be configured to, in response to detecting a subsequent connection of the treatment source to the wound dressing and in response to a determination that the identification data does not match the pairing data or that the timer has reached the lifetime threshold, disallow the treatment source to supply the stimulation to the wound dressing. The control circuitry can be configured to deactivate operation of the wound dressing in response to a determination that the timer has reached the lifetime threshold.

The wound treatment apparatus of any of the preceding paragraphs and/or any of the apparatuses, systems, or devices disclosed herein can include one or more of the following features. The treatment source can include at least one of a source of vibrational energy, a light source, or a negative pressure source. The control circuitry can be configured to deactivate operation of the wound dressing by application of an electrical signal configured to sever at least one electrical connection in the wound dressing. The electrical signal can be configured to deactivate a fuse of the wound dressing. The control circuitry can be configured to deactivate operation of the wound dressing by at least partially erasing a memory of the wound dressing. The control circuitry can be configured to deactivate operation of the wound dressing by storing a deactivation code in a memory of the wound dressing. The control circuitry can be configured to cause the wound dressing to store the pairing data in a memory of the wound dressing. The control circuitry can be configured to start the timer configured to monitor operational lifetime of the wound dressing responsive to a verification of at least one of: that treatment to the wound is being provided by the one or more electronic components or that application of negative pressure to the wound has been detected. The verification can be performed by the control circuitry. The verification that the treatment to the wound is being provided by the one or more electronic components can be based on at least one of: detecting a temperature increase of at least one of the one or more electronic components, detecting a temperature increase of tissue in at least a portion of the wound, detecting feedback electrical current in at least one of the one or more electronic components, or detecting variation in impedance of wound tissue.

The wound treatment apparatus of any of the preceding paragraphs and/or any of the apparatuses, systems, or devices disclosed herein can include one or more of the following features. The control circuitry can be configured to, in response to detecting an initial connection of the treatment source to another wound dressing comprising another one or more electronic components, provide treatment to the wound or to another wound in response to the stimulation received from the treatment source. The control circuitry can be configured to, in response to detecting an initial connection of the treatment source to another wound dressing comprising another one or more electronic components, communicate another pairing data to the another wound dressing. The control circuitry can be configured to, in response to detecting an initial connection of the treatment source to another wound dressing comprising another one or more electronic components, start another timer configured to monitor operational lifetime of the another wound dressing. The control circuitry can be configured to, in response to detecting a subsequent connection of the treatment source to the wound dressing or to the another wound dressing, request and receive from the wound dressing or the another wound dressing the identification data. The control circuitry can be configured to, in response to detecting a subsequent connection of the treatment source to the wound dressing or to the another wound dressing and in response to the determination that the identification data matches the pairing data and that the timer has not reached the lifetime threshold, cause the treatment source to supply the stimulation to the wound dressing. The control circuitry can be configured to, in response to detecting a subsequent connection of the treatment source to the wound dressing or to the another wound dressing and in response to a determination that the identification data matches the another pairing data and that the another timer has not reached another lifetime threshold, cause the treatment source to supply the stimulation to the another wound dressing. The control circuitry can be configured to, in response to detecting a subsequent connection of the treatment source to the wound dressing or to the another wound dressing, disallow the treatment source to supply the stimulation to at least one of the wound dressing or the another wound dressing in response to a determination of at least one of: that the identification data does not match the pairing data or the another pairing data, that the identification data matches the pairing data and that the timer has reached the lifetime threshold, or that the identification data matches the another pairing data and that the another timer has reached the another lifetime threshold. The control circuitry can be configured to, in response to detecting a subsequent connection of the treatment source to the wound dressing or to the another wound dressing, deactivate operation of the another wound dressing in response to a determination that the another timer has reached the another lifetime threshold.

The wound treatment apparatus of any of the preceding paragraphs and/or any of the apparatuses, systems, or devices disclosed herein can include one or more of the following features. The lifetime threshold and the another lifetime threshold can be the same. The control circuitry can be configured to cause the another wound dressing to store the another pairing data.

The wound treatment apparatus of any of the preceding paragraphs and/or any of the apparatuses, systems, or devices disclosed herein can include one or more of the following features. A kit can include the wound treatment apparatus and the wound dressing or the another wound dressing. The one or more electronic components can include at least one ultrasonic transducer configured to apply vibrational energy to the wound. The wound dressing can include a connector configured to connect the wound dressing to the treatment source. The fuse can be positioned in the connector.

A wound treatment apparatus can include a control circuitry for applying wound treatment. The control circuitry can be configured to, in response to detecting an initial connection of a treatment source of the apparatus for applying wound treatment to a wound dressing covering a wound, communicate pairing data to a wound dressing. The control circuitry can be configured to, in response to detecting an initial connection of a treatment source of the apparatus for applying wound treatment to a wound dressing covering a wound, start a timer configured to monitor operational lifetime of the wound dressing. The treatment source can be configured to supply stimulation that causes the wound dressing to provide treatment to the wound. The control circuitry can be configured to, in response to detecting a subsequent connection of the treatment source to the wound dressing, receive identification data from the wound dressing. The control circuitry can be configured to, in response to detecting a subsequent connection of the treatment source to the wound dressing and in response to determining that the identification data matches the pairing data and that the timer has not reached a lifetime threshold, cause the treatment source to supply the stimulation to the wound dressing. The control circuitry can be configured to, in response to detecting a subsequent connection of the treatment source to the wound dressing and in response to determining that the identification data does not match the pairing data or that the timer has reached the lifetime threshold, disallow the treatment source to supply the stimulation to the wound dressing. The control circuitry can be configured to deactivate operation of the wound dressing in response to determining that the timer has reached the lifetime threshold.

The wound treatment apparatus of any of the preceding paragraphs and/or any of the apparatuses, systems, or devices disclosed herein can include one or more of the following features. The treatment source can include at least one of a source of vibrational energy, a light source, or a negative pressure source. The control circuitry can be configured to deactivate operation of the wound dressing by application of an electrical signal configured to sever at least one electrical connection in the wound dressing. The electrical signal can be configured to deactivate a fuse of the wound dressing. The control circuitry can be configured to deactivate operation of the wound dressing by at least one of: 1) at least partially erasing a memory of the wound dressing or 2) storing a deactivation code in the memory of the wound dressing. The control circuitry can be configured to cause the wound dressing to store the pairing data in a memory of the wound dressing.

The wound treatment apparatus of any of the preceding paragraphs and/or any of the apparatuses, systems, or devices disclosed herein can include one or more of the following features. The control circuitry can be configured to, in response to detecting an initial connection of the treatment source to another wound dressing, provide treatment to the wound or to another wound in response to the stimulation received from the treatment source. The control circuitry can be configured to, in response to detecting an initial connection of the treatment source to another wound dressing, communicate another pairing data to the another wound dressing. The control circuitry can be configured to, in response to detecting an initial connection of the treatment source to another wound dressing, start another timer configured to monitor operational lifetime of the another wound dressing. The control circuitry can be configured to, in response to detecting a subsequent connection of the treatment source to the wound dressing or to the another wound dressing, request and receive from the wound dressing or the another wound dressing the identification data. The control circuitry can be configured to, in response to detecting a subsequent connection of the treatment source to the wound dressing or to the another wound dressing and in response to determining that the identification data matches the pairing data and that the timer has not reached the lifetime threshold, cause the treatment source to supply the stimulation to the wound dressing. The control circuitry can be configured to, in response to detecting a subsequent connection of the treatment source to the wound dressing or to the another wound dressing and in response to determining that the identification data matches the another pairing data and that the another timer has not reached another lifetime threshold, cause the treatment source to supply the stimulation to the another wound dressing. The control circuitry can be configured to, in response to detecting a subsequent connection of the treatment source to the wound dressing or to the another wound dressing, disallow the treatment source to supply the stimulation to at least one of the wound dressing or the another wound dressing in response to determining at least one of: that the identification data does not match the pairing data or the another pairing data, that the identification data matches the pairing data and that the timer has reached the lifetime threshold, or that the identification data matches the another pairing data and that the another timer has reached the another lifetime threshold. The control circuitry can be configured to deactivate operation of the another wound dressing in response to determining that the another timer has reached the another lifetime threshold.

The wound treatment apparatus of any of the preceding paragraphs and/or any of the apparatuses, systems, or devices disclosed herein can include one or more of the following features. The lifetime threshold and the another lifetime threshold can be the same. The control circuitry can be configured to cause the another wound dressing to store the another pairing data. The wound dressing can be configured to apply vibrational energy to the wound. The another wound dressing can be configured to apply vibrational energy to the another wound.

Disclosed are methods of operating a wound treatment device of any of the preceding paragraphs and/or any of the apparatuses, systems, or devices disclosed herein.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIG. 1 illustrates a reduced pressure wound therapy system.

FIG. 2 illustrates a therapeutic ultrasound wound treatment apparatus.

FIG. 3 illustrates a therapeutic ultrasound wound treatment apparatus.

FIG. 4A illustrates a schematic of a therapeutic ultrasound wound treatment apparatus.

FIGS. 4B and 4C illustrates a therapeutic ultrasound wound treatment apparatuses.

FIG. 5 illustrates a wound dressing that may be used with an ultrasound wound treatment apparatus.

FIGS. 6A, 6B, and 6C illustrate a therapeutic ultrasound wound treatment apparatus.

FIG. 7 is a flowchart of an example process for managing operation of a wound dressing.

### DETAILED DESCRIPTION

Embodiments disclosed herein relate to systems and methods of managing a limited use wound dressing or device for monitoring or treating a wound. A limited use wound dressing may be a wound dressing that is configured to be in use for a limited duration of time. A limited use device can be a device that is configured to be in use for a limited duration of time. Throughout this specification reference is made to a wound. The term wound is to be broadly construed and encompasses open and closed wounds in which skin is torn, cut or punctured or where trauma causes a contusion, or any other superficial or other conditions or imperfections on the skin of a patient or otherwise that benefit from pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, bums, diabetic ulcers, pressure ulcers, stoma, surgical wounds, trauma and venous ulcers or the like.

Embodiments of systems and methods disclosed herein can be used with treatment systems, such as topical negative pressure (“TNP”) or reduced pressure therapy systems, ultrasound therapy systems, light therapy systems, surgical debridement systems (such as, Versajet sold by Smith & Nephew), or any type of treatment systems. Additionally or alternatively, embodiments of systems and methods disclosed herein can be used with monitoring systems, such as a monitoring system that includes a wound dressing supporting one or more sensors and a control device configured to communicate with the wound dressing. For instance, such monitoring system is disclosed in U.S. Patent Application Publication No. 2019/0290496, which is incorporated by reference in its entirety.

The systems and methods disclosed herein can be used to manage limited use wound dressings that include (such as, support) one or more electronic components. The systems and methods disclosed herein can include control circuitry that causes provision of therapy to via a wound dressing or communicate with the wound dressing. The control circuitry can disable the wound dressing after a limited number of uses. The systems and methods disclosed herein can enable a wound dressing to be used with a limited set of control circuitry. This reduces the risk of a potential infection or other complications.

Briefly, negative pressure wound therapy assists in the closure and healing of many forms of “hard to heal” wounds by reducing tissue oedema, encouraging blood flow and granular tissue formation, or removing excess exudate and can reduce bacterial load (and thus infection risk). In addition, the therapy allows for less disturbance of a wound leading to more rapid healing. TNP therapy systems can also assist in the healing of surgically closed wounds by removing fluid. TNP therapy can help to stabilize the tissue in the apposed position of closure. A further beneficial use of TNP therapy can be found in grafts and flaps where removal of excess fluid is important and close proximity of the graft to tissue is required in order to ensure tissue viability.

As used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects pressure that is X mmHg below 760 mmHg or, in other words, a pressure of (760−X) mmHg. In addition, negative pressure that is “less” or “smaller” than X mmHg corresponds to pressure that is closer to atmospheric pressure (for example, −40 mmHg is less than ˜60 mmHg). Negative pressure that is “more” or “greater” than −X mmHg corresponds to pressure that is further from atmospheric pressure (for example, −80 mmHg is more than ˜60 mmHg). In some cases, local ambient atmospheric pressure is used as a reference point, and such local atmospheric pressure may not necessarily be, for example, 760 mmHg.

Systems and methods disclosed herein can be used with other types of treatment in addition to or instead of reduced pressure therapy, such as irrigation, ultrasound, heat or cold, neuro stimulation, or the like. In some cases, disclosed systems and methods can be used for wound monitoring without application of additional therapy. Systems and methods disclosed herein can be used in conjunction with a dressing, including with compression dressing, reduced pressure dressing, or the like.

The systems and methods disclosed herein may be used in combination with clothing; for example, by incorporating a transducer (e.g., an ultrasound transducer) within layers of clothing. Non-limiting examples of clothing herein include shirts, pants, trousers, dresses, undergarments, outer-garments, gloves, shoes, hats, and other suitable garments. The systems and methods disclosed herein may be incorporated into cushioning or bed padding, such as within a hospital bed, to monitor patient characteristics, such as any characteristic disclosed herein. A disposable film containing such sensors can be placed over the hospital bedding and removed/replaced as needed.

The systems and methods disclosed herein may be utilized in rehabilitation devices and treatments, including sports medicine. For example, the systems and methods disclosed herein may be used in braces, sleeves, wraps, supports, and other suitable items. The systems and methods disclosed herein may be incorporated into implantable devices, such as implantable orthopedic implants, including flexible implants. Such implants may be configured to treat tissue surrounding the implant site. An internal source may also provide power for such an implant. Treatment of such wounds can be performed using traditional wound care, wherein a dressing can be applied to the wound to facilitate and promote healing of the wound. Some methods relate to methods of manufacturing a wound dressing comprising providing a wound dressing as disclosed herein. The wound dressings that may be utilized in conjunction with the disclosed technology include any known dressing in the art. The technology is applicable to negative pressure therapy treatment as well as non-negative pressure therapy treatment.

A wound dressing may include one or more absorbent layer(s). The absorbent layer may be a foam or a superabsorbent. Wound dressings may comprise a dressing layer including a polysaccharide or modified polysaccharide, a polyvinylpyrrolidone, a polyvinyl alcohol, a polyvinyl ether, a polyurethane, a polyacrylate, a polyacrylamide, collagen, or gelatin or mixtures thereof. Dressing layers comprising the polymers listed are known in the art as being useful for forming a wound dressing layer for either negative pressure therapy or non-negative pressure therapy. The polymer matrix may be a polysaccharide or modified polysaccharide. The polymer matrix may be a cellulose. Cellulose material may include hydrophilically modified cellulose such as methyl cellulose, carboxymethyl cellulose (CMC), carboxymethyl cellulose (CEC), ethyl cellulose, propyl cellulose, hydroxyethyl cellulose, hydroxypropyl cellulose, hydroxypropylmethyl cellulose, carboxyethyl sulphonate cellulose, cellulose alkyl sulphonate, or mixtures thereof.

Cellulose material may be cellulose alkyl sulphonate. The alkyl moiety of the alkyl sulphonate substituent group may have an alkyl group having 1 to 6 carbon atoms, such as methyl, ethyl, propyl, or butyl. The alkyl moiety may be branched or unbranched, and hence suitable propyl sulphonate substituents may be 1- or 2-methyl-ethylsulphonate. Butyl sulphonate substituents may be 2-ethyl-ethylsulphonate, 2,2-dimethyl-ethylsulphonate, or 1,2-dimethyl-ethylsulphonate. The alkyl sulphonate substituent group may be ethyl sulphonate. The cellulose alkyl sulphonate is described in PCT Publication No. WO 2010/061225, filed Nov. 27, 2009; U.S. Patent Application Publication No. 2016/0114074, filed Nov. 16, 2015; U.S. Patent Application Publication No. 2006/0142560, filed Dec. 29, 2004; and U.S. Pat. No. 5,703,225, filed Dec. 13, 1995, each of which are hereby incorporated by reference in their entirety.

Cellulose alkyl sulfonates may have varying degrees of substitution, the chain length of the cellulose backbone structure, and the structure of the alkyl sulfonate substituent. Solubility and absorbency are largely dependent on the degree of substitution: as the degree of substitution is increased, the cellulose alkyl sulfonate becomes increasingly soluble. It follows that, as solubility increases, absorbency increases. A wound dressing may include a top or cover layer. The thickness of the wound dressing disclosed herein may be between 1 to 20, or 2 to 10, or 3 to 7 mm.

The disclosed technology may be used in conjunction with a non-negative pressure dressing. A non-negative pressure wound dressing suitable for providing protection at a wound site may comprise: an absorbent layer for absorbing wound exudate and an obscuring element for at least partially obscuring a view of wound exudate absorbed by the absorbent layer in use. The obscuring element may be partially translucent, and the obscuring element may be a masking layer.

The non-negative pressure wound dressing may further comprise a region in or adjacent the obscuring element for allowing viewing of the absorbent layer. For example, the obscuring element layer may be provided over a central region of the absorbent layer and not over a border region of the absorbent layer. The obscuring element may be hydrophilic material or may be coated with a hydrophilic material. The obscuring element may comprise a three-dimensional knitted spacer fabric. The spacer fabric is known in the art and may include a knitted spacer fabric layer. The obscuring element may further comprise an indicator for indicating the need to change the dressing. The obscuring element may be a layer at least partially over the absorbent layer, further from a wound site than the absorbent layer in use. The non-negative pressure wound dressing may further comprise a plurality of openings in the obscuring element for allowing fluid to move therethrough. The obscuring element may comprise, or may be coated with, a material having size-exclusion properties for selectively permitting or preventing passage of molecules of a predetermined size or weight. The obscuring element may be configured to at least partially mask light radiation having wavelength of 600 nm and less. The obscuring element may be configured to reduce light absorption by 50% or more. The obscuring element may be configured to yield a CIE L* value of 50 or more, and optionally 70 or more. The obscuring element may be configured to yield a CIE L* value of 70 or more.

The non-negative pressure wound dressing may further include at least one of a wound contact layer, a foam layer, an odor control element, a pressure-resistant layer and a cover layer. The cover layer may be present, and the cover layer may be a translucent film. Typically, the translucent film has a moisture vapour permeability of 500 g/m2/24 hours or more. The translucent film may be a bacterial barrier. The non-negative pressure wound dressing as disclosed herein may include the wound contact layer and the absorbent layer overlies the wound contact layer. The wound contact layer carries an adhesive portion for forming a substantially fluid tight seal over the wound site. The non-negative pressure wound dressing as disclosed herein may comprise the obscuring element and the absorbent layer being provided as a single layer. The non-negative pressure wound dressing disclosed herein may include the foam layer, and the obscuring element is of a material comprising components that may be displaced or broken by movement of the obscuring element.

The non-negative pressure wound dressing may include an odor control element. In some cases, the dressing may not include an odor control element. When present, the odor control element may be dispersed within or adjacent the absorbent layer or the obscuring element. Alternatively, when present the odor control element may be provided as a layer sandwiched between the foam layer and the absorbent layer.

The disclosed technology for a non-negative pressure wound dressing can include a method of manufacturing a wound dressing, comprising: providing an absorbent layer for absorbing wound exudate; and providing an obscuring element for at least partially obscuring a view of wound exudate absorbed by the absorbent layer in use.

The non-negative pressure wound dressing may be suitable for providing protection at a wound site, comprising: an absorbent layer for absorbing wound exudate; and a shielding layer provided over the absorbent layer, and further from a wound-facing side of the wound dressing than the absorbent layer. The shielding layer may be provided directly over the absorbent layer. The shielding layer can include a three-dimensional spacer fabric layer. The shielding layer increases the area over which a pressure applied to the dressing is transferred by 25% or more or the initial area of application. For example the shielding layer increases the area over which a pressure applied to the dressing is transferred by 50% or more, and optionally by 100% or more, and optionally by 200% or more. The shielding layer may comprise 2 or more sub-layers, wherein a first sub-layer comprises through holes and a further sub-layer comprises through holes and the through holes of the first sub-layer are offset from the through holes of the further sub-layer. The non-negative pressure wound dressing as disclosed herein may further comprise a permeable cover layer for allowing the transmission of gas and vapour therethrough, the cover layer provided over the shielding layer, wherein through holes of the cover layer are offset from through holes of the shielding layer. The non-negative pressure wound dressing may be suitable for treatment of pressure ulcers. A more detailed description of the non-negative pressure dressing disclosed hereinabove is provided in PCT Publication No. WO 2013/007973, filed Jul. 12, 2012, which is hereby incorporated by reference in its entirety. The non-negative pressure wound dressing may be a multi-layered wound dressing comprising: a fibrous absorbent layer for absorbing exudate from a wound site; and a support layer configured to reduce shrinkage of at least a portion of the wound dressing.

The multi-layered wound dressing disclosed herein, further can include a liquid impermeable film layer, wherein the support layer is located between the absorbent layer and the film layer. The support layer disclosed herein may comprise a net. The net may comprise a geometric structure having a plurality of substantially geometric apertures extending therethrough. The geometric structure may for example comprise a plurality of bosses substantially evenly spaced and joined by polymer strands to form the substantially geometric apertures between the polymer strands. The net may be formed from high density polyethylene. The apertures may have an area from 0.005 to 0.32 mm2. The support layer may have a tensile strength from 0.05 to 0.06 Nm. The support layer may have a thickness of from 50 to 150 μm.

The support layer can be located directly adjacent the absorbent layer. Typically, the support layer is bonded to fibers in a top surface of the absorbent layer. The support layer may further comprise a bonding layer, wherein the support layer is heat laminated to the fibers in the absorbent layer via the bonding layer. The bonding layer may comprise a low melting point adhesive such as ethylene-vinyl acetate adhesive. The multi-layered wound dressing disclosed herein further can include an adhesive layer attaching the film layer to the support layer.

The multi-layered wound dressing disclosed herein further can include a wound contact layer located adjacent the absorbent layer for positioning adjacent a wound. The multi-layered wound dressing may further comprise a fluid transport layer between the wound contact layer and the absorbent layer for transporting exudate away from a wound into the absorbent layer. A more detailed description of the multi-layered wound dressing disclosed hereinabove is provided in PCT Patent Publication No. WO 2018/07872, filed on Oct. 24, 2017; and PCT Patent Publication No. WO 2017/077154, each of which are hereby incorporated by reference in their entirety.

The disclosed technology may be incorporated in a wound dressing comprising a vertically lapped material comprising: a first layer of an absorbing layer of material, and a second layer of material, wherein the first layer being constructed from at least one layer of non-woven textile fibers, the non-woven textile fibers being folded into a plurality of folds to form a pleated structure. The wound dressing further can include a second layer of material that is temporarily or permanently connected to the first layer of material. Typically the vertically lapped material has been slitted.

The first layer can include aa pleated structure having a depth determined by the depth of pleats or by the slitting width. The first layer of material may be a moldable, lightweight, fiber-based material, blend of material or composition layer. The first layer of material may comprise one or more of manufactured fibers from synthetic, natural or inorganic polymers, natural fibers of a cellulosic, proteinaceous or mineral source.

The wound dressing may comprise two or more layers of the absorbing layer of material vertically lapped material stacked one on top of the other, wherein the two or more layers have the same or different densities or composition. The wound dressing may comprise only one layer of the absorbing layer of material vertically lapped material. The absorbing layer of material is a blend of natural or synthetic, organic or inorganic fibers, and binder fibers, or bicomponent fibers typically PET with a low melt temperature PET coating to soften at specified temperatures and to act as a bonding agent in the overall blend. The absorbing layer of material may be a blend of 5 to 95% thermoplastic polymer, and 5 to 95 wt % of a cellulose or derivative thereof.

The wound dressing disclosed herein has a second layer comprises a foam or a dressing fixative. The foam may be a polyurethane foam. The polyurethane foam may have an open or closed pore structure. The dressing fixative may include bandages, tape, gauze, or backing layer.

The wound dressing as disclosed herein may include the absorbing layer of material connected directly to a second layer by lamination or by an adhesive, and the second layer is connected to a dressing fixative layer. The adhesive may be an acrylic adhesive, or a silicone adhesive. The wound dressing as disclosed herein further can include a layer of a superabsorbent fiber, or a viscose fiber or a polyester fiber. The wound dressing as disclosed herein further can include a backing layer. The backing layer may be a transparent or opaque film. Typically the backing layer comprises a polyurethane film (typically a transparent polyurethane film). A more detailed description of the multi-layered wound dressing disclosed hereinabove is provided in GB Patent Application No. 1621057.7, filed Dec. 12, 2016; and GB Patent Application No. 1709987.0, filed Jun. 22, 2017; and GB Patent Application No. 1817052.2, filed Oct. 19, 2018, each of which are hereby incorporated by reference in their entirety

The non-negative pressure wound dressing may comprise an absorbent component for a wound dressing, the component comprising a wound contacting layer comprising gel forming fibers bound to a foam layer, wherein the foam layer is bound directly to the wound contact layer by an adhesive, polymer based melt layer, by flame lamination or by ultrasound. The absorbent component may be in a sheet form. The wound contacting layer may comprise a layer of woven or non-woven or knitted gel forming fibers. The foam layer may be an open cell foam, or closed cell foam, typically an open cell foam. The foam layer is a hydrophilic foam.

The wound dressing may comprise the component that forms an island in direct contact with the wound surrounded by periphery of adhesive that adheres the dressing to the wound. The adhesive may be a silicone or acrylic adhesive, typically a silicone adhesive. The wound dressing may be covered by a film layer on the surface of the dressing furthest from the wound. A more detailed description of the wound dressing of this type hereinabove is provided in PCT Publication No. WO 2011058311A1, filed Nov. 10, 2010, as International Application No. PCT/GB2010/002071, each of which is hereby incorporated by reference in their entirety.

The non-negative pressure wound dressing may comprise a multi layered wound dressing for use on wounds producing high levels of exudate, characterized in that the dressing comprising: a transmission layer having an MVTR of at least 300 gm2/24 hours, an absorbent core comprising gel forming fibers capable of absorbing and retaining exudate, a wound contacting layer comprising gel forming fibers which transmits exudate to the absorbent core and a keying layer positioned on the absorbent core, the absorbent core and wound contacting layer limiting the lateral spread of exudate in the dressing to the region of the wound. The wound dressing may be capable of handling at least 6 g (or 8 g and 15 g) of fluid per 10 cm2 of dressing in 24 hours. The wound dressing may comprise gel forming fibers that are chemically modified cellulosic fibers in the form of a fabric. The fibers may include carboxymethylated cellulose fibers, typically sodium carboxymethylcellulose fiber. The wound dressing may comprise a wound contact layer with a lateral wicking rate from 5 mm per minute to 40 mm per minute. The wound contact layer may have a fiber density between 25 gm2 and 55 gm2, such as 35 gm2. The absorbent core may have an absorbency of exudate of at least 10 g/g, and typically a rate of lateral wicking of less the 20 mm per minute. The absorbent core may have a blend in the range of up to 25% cellulosic fibers by weight and 75% to 100% gel forming fibers by weight.

Alternatively, the absorbent core may have a blend in the range of up to 50% cellulosic fibers by weight and 50% to 100% gel forming fibers by weight. For example the blend is in the range of 50% cellulosic fibers by weight and 50% gel forming fibers by weight. The fiber density in the absorbent core may be between 150 gm2 and 250 gm2, or about 200 gm2. The wound dressing when wet may have shrinkage that is less than 25% or less than 15% of its original size/dimension. The wound dressing may comprise a transmission layer and the layer is a foam. The transmission layer may be a polyurethane foam laminated to a polyurethane film.

The wound dressing may comprise one or more layers selected from the group comprising a soluble medicated film layer; an odor-absorbing layer; a spreading layer and an additional adhesive layer. The wound dressing may be 2 mm and 4 mm thick. The wound dressing may be characterized in that the keying layer bonds the absorbent core to a neighboring layer. The keying layer may be positioned on either the wound facing side of the absorbent core or the non-wound facing side of the absorbent core. The keying layer can be positioned between the absorbent core and the wound contact layer. The keying layer can be a polyamide web. A more detailed description of the wound dressing of this type hereinabove is provided in PCT Publication No. WO 2005079718, filed Feb. 11, 2005, as International Application No. PCT/GB2005/000517, each of which is hereby incorporated by reference in their entirety.

The non-negative pressure wound dressing may be a compression bandage. Compression bandages are known for use in the treatment of oedema and other venous and lymphatic disorders, e.g., of the lower limbs. A compression bandage system can typically employ multiple layers including a padding layer between the skin and the compression layer or layers. The compression bandage may be useful for wounds such as handling venous leg ulcers. The compression bandage may comprise a bandage system comprising an inner skin facing layer and an elastic outer layer, the inner layer comprising a first ply of foam and a second ply of an absorbent nonwoven web, the inner layer and outer layer being sufficiently elongated so as to be capable of being wound about a patient's limb. A compression bandage of this type is disclosed in PCT Publication No. WO 1999/58090, filed Nov. 19, 1999, which is hereby incorporated by reference in its entirety. The compression bandage system comprises: a) an inner skin facing, elongated, elastic bandage comprising: (i) an elongated, elastic substrate, and (ii) an elongated layer of foam, said foam layer being affixed to a face of said substrate and extending 33% or more across said face of substrate in transverse direction and 67% or more across said face of substrate in longitudinal direction; and b) an outer, elongated, self-adhering elastic bandage; said bandage having a compressive force when extended; wherein, in use, said foam layer of the inner bandage faces the skin and the outer bandage overlies the inner bandage. A compression bandage of this type is disclosed in PCT Publication No. WO 2006/110527, filed Apr. 5, 2006, which is hereby incorporated by reference in its entirety. Other compression bandage systems can be used such as those disclosed in U.S. Pat. No. 6,759,566, filed Jul. 13, 1998; and U.S. Patent Application Publication No. 2002/0099318, filed May 14, 1998, each of which are hereby incorporated by reference in their entirety.

The systems and methods disclosed herein relate to the use of a limited use wound dressing. The limited use wound dressing may include one or more electrical components in order to provide a therapy function to a wound. In order to provide the therapy function, the limited use wound dressing may be connected to control circuitry that causes the wound dressing to perform wound therapy (e.g., TNP therapy, ultrasound therapy, compression, light therapy, etc.). The control circuitry can drive the limited use wound dressing by providing a drive signal to the limited use wound dressing that causes the wound dressing to perform wound therapy. Upon connecting the control circuitry and the limited use wound dressing, the control circuitry and the limited use wound dressing can be paired such that the limited use wound dressing only performs wound therapy when connected to the particular, authorized control circuitry. Further, each limited use wound dressing may be paired a limited number of times or to a limited number of control circuitries. Additionally, the control circuitry may be configured to drive the limited use wound dressing for a particular, configurable period of time. Prior to enabling the wound dressing to perform wound therapy, the driving circuit may be configured to verify that the control circuitry and the limited use wound dressing have been paired and that the timer has not reached a lifetime threshold corresponding a threshold pairing time for the limited use wound dressing and the driving circuit. Based upon reaching the lifetime threshold, the control circuitry may disable the limited use wound dressing. For example, the control circuitry may disable the limited use wound dressing via a fuse of the limited use wound dressing.

### Wound Therapy Systems

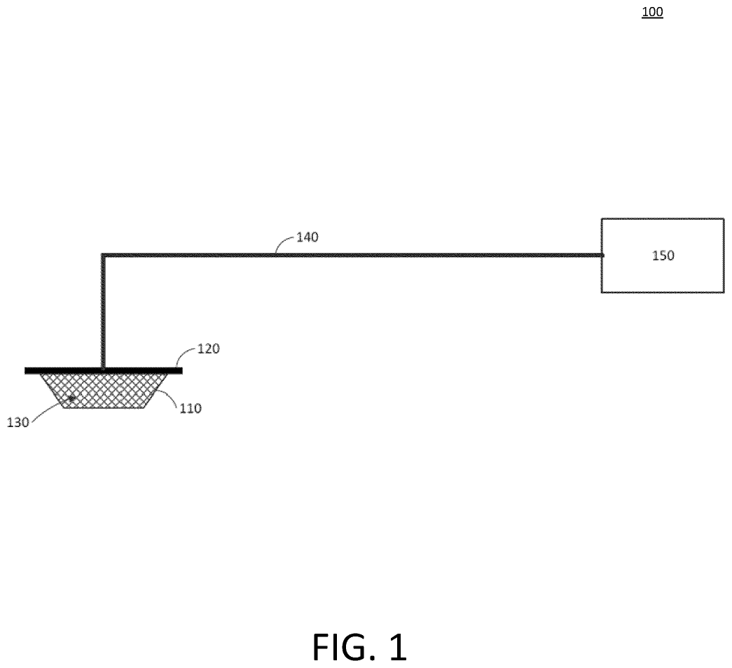

FIG. 1 illustrates a wound treatment system 100 comprising a wound filler 130 placed inside a wound cavity 110, the wound cavity sealed by a wound cover 120. The wound treatment system 100 may be a negative or reduced pressure wound treatment (or TNP) system 100. The wound filler 130 in combination with the wound cover 120 can be referred to as wound dressing. The wound dressing can include one or more electrical components in order to enable the wound dressing to provide a particular function (e.g., a pressure function). A single or multi lumen tube or conduit 140 is connected the wound cover 120 with a pump assembly 150 configured to supply reduced pressure. The wound cover 120 can be in fluidic communication with the wound cavity 110. The pump assembly can be a canisterless pump assembly (meaning that exudate is collected in the wound dressing or is transferred via tube 140 for collection to another location). However, the pump assembly can be configured to include or support a canister. Additionally, the pump assembly can be mounted to or supported by the dressing, or adjacent to the dressing.

The wound filler 130 can be any suitable type, such as hydrophilic or hydrophobic foam, gauze, inflatable bag, and so on. The wound filler 130 can be conformable to the wound cavity 110 such that it substantially fills the cavity. The wound cover 120 can provide a substantially fluid impermeable seal over the wound cavity 110. The wound cover 120 can have a top side and a bottom side, and the bottom side adhesively (or in any other suitable manner) seals with wound cavity 110. The conduit 140 or lumen or any other conduit or lumen disclosed herein can be formed from polyurethane, PVC, nylon, polyethylene, silicone, or any other suitable material.

The wound cover 120 can have a port (not shown) configured to receive an end of the conduit 140. For example, the port can be Renays Soft Port available from Smith & Nephew. The conduit 140 can otherwise pass through or under the wound cover 120 to supply reduced pressure to the wound cavity 110 so as to maintain a target or desired level of reduced pressure in the wound cavity. The conduit 140 can be any suitable article configured to provide at least a substantially sealed fluid flow pathway between the pump assembly 150 and the wound cover 120, so as to supply the reduced pressure provided by the pump assembly 150 to wound cavity 110.

The wound cover 120 and the wound filler 130 can be provided as a single article or an integrated single unit. In some cases, no wound filler is provided and the wound cover by itself may be considered the wound dressing. The wound dressing may then be connected, via the conduit 140, to a source of negative pressure, such as the pump assembly 150. The pump assembly 150 can be miniaturized and portable, although larger conventional pumps such can also be used.

The wound cover 120 can be located over a wound site to be treated. The wound cover 120 can form a substantially sealed cavity or enclosure over the wound site. The wound cover 120 can be configured to have a film having a high water vapor permeability to enable the evaporation of surplus fluid, and can have a superabsorbing material contained therein to safely absorb wound exudate.

The system can operate without the use of an exudate canister. The system can be configured to support an exudate canister. Configuring the pump assembly 150 and tube 140 so that the tube 140 can be quickly and easily removed from the pump assembly 150 can facilitate or improve the process of dressing or pump changes, if necessary. The pump can be configured to have any suitable connection between the tube and the pump.

The pump assembly 150 can be configured to deliver negative pressure of approximately −80 mmHg, or between about −20 mmHg and 200 mmHg in some implementations. Note that these pressures are relative to normal ambient atmospheric pressure thus, −200 mmHg would be about 560 mmHg in practical terms. The pressure range can be between about −40 mmHg and −150 mmHg. Alternatively a pressure range of up to −75 mmHg, up to −80 mmHg or over −80 mmHg can be used. Also a pressure range of below −75 mmHg can be used. Alternatively a pressure range of over approximately −100 mmHg, or even 150 mmHg, can be supplied by the pump assembly 150.

In operation, the wound filler 130 is inserted into the wound cavity 110 and wound cover 120 is placed so as to seal the wound cavity 110. The pump assembly 150 provides a source of a negative pressure to the wound cover 120, which is transmitted to the wound cavity 110 via the wound filler 130. Fluid (e.g., wound exudate) is drawn through the conduit 140, and can be stored in a canister. The fluid can be absorbed by the wound filler 130 or one or more absorbent layers (not shown).

Wound dressings that may be utilized with the pump assembly include Renasys-F, Renasys-G, Renasys AB, and Pico Dressings available from Smith & Nephew. Further description of such wound dressings and other components of a negative pressure wound therapy system that may be used with the pump assembly are found in U.S. Patent Application Publication No. 2011/0213287, filed Sep. 20, 2010; and U.S. Patent Application Publication No. 2011/0282309, filed Apr. 21, 2011; and U.S. Patent Application Publication No. 2012/0116334, filed Dec. 22, 2010; and U.S. Patent Application Publication No. 2012/0136325, filed Nov. 2, 2011; and U.S. Patent Application Publication No. 2013/0110058, filed Nov. 2, 2011, each of which are hereby incorporated by reference in their entirety. Other suitable wound dressings can be utilized.

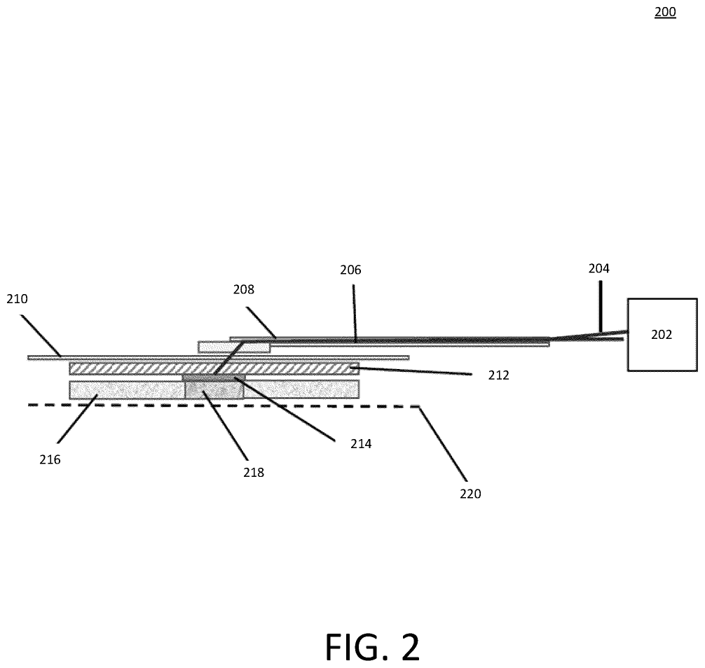

FIG. 2 depicts a therapeutic ultrasound wound treatment apparatus 200 for use in treating a wound. The dressing used by the apparatus 200 cam be similar to the dressing described in connection with FIG. 1. A suitable signal may be delivered to control circuitry associated with the dressing from an electrical signal generator 202 (e.g., a driving unit), via wires 204, in order to drive/power the control circuitry. Based upon receiving the signal, the control circuitry may provide stimulation to the wound dressing. One of skill in the art will understand that the signal may have any parameters such as timing, intensity, and frequency disclosed herein this section or elsewhere in the specification. The electrical signal generator may be of any suitable type, for example, the EXOGEN generator manufactured by Bioventus. The wires may be contained within a channel 206 contained within a port 208, such as the soft port described in relation to FIG. 1. The channel 206 may have 2 layers of spacer to provide space for the wires to pass. The port may be connected to a cover layer 210, which may be adhered to the port via a constructional adhesive. The cover layer can overlay an absorbent layer 212, the absorbent layer constructed from any material disclosed herein this section or elsewhere in the specification, for example a cellulose material with embedded superabsorbent particles. An transducer 214 may be located beneath the absorbent layer. The transducer 214 may be an ultrasonic transducer. The transducer may be of any suitable type, such as a piezoelectric transducer, a capacitive transducer, and/or any suitable transducer such as those described in PCT Publication No. WO 1999/056829, filed May 6, 1999; and PCT Publication No. WO 1999/048621, filed Sep. 30, 1999; and U.S. Pat. No. 5,904,659, filed Jul. 1, 1997, each of which are hereby incorporated by reference in their entirety. Once the transducer is stimulated by a suitable electrical signal from the signal generator 202, the transducer will emit a therapeutic signal. The therapeutic signal provided by the transducer may be highly variable in terms of timing of pulsation, frequency, and intensity. Further details regarding the therapeutic signal are provided below. The therapeutic wound treatment apparatuses disclosed herein may comprise about: one, two, three, four, five, ten, fifteen, 25, 50, 75, 100, 150, 200, 300 or more transducers. The transducers may be organized into a grid, such as a grid contained within a flexible substrate.

The signal can be delivered to the wounded surface with sufficient intensity to stimulate healing. Therefore, it is desirable that a delivery pathway be available for the signal to be effectively transmitted to the surface. Such a delivery pathway may be provided by a delivery layer 216 comprising a transmission portion 218. The delivery layer may comprise foam surrounding the transmission portion 218. For example, the delivery layer may be constructed from a polyurethane foam or any suitable material such as disclosed herein this section or elsewhere in the specification. The delivery layer may comprise one, two, three, four, five, ten, fifteen, or more transmission portions. The transmission portions may have any suitable shape, such as a column, a pillar, a cuboid, or a rectangular parallelepiped. The transmission portion may be in the form of a strip or a series of layered strips adjacent to a transducer. Any shape may be suitable for the transmission portion, provided that there is a continuous transmission portion “line-of-sight” between the transducer and the wounded tissue. Transmission can be sensitive to the medium used for transmission, therefore without a path to the wound of transmission material, the signal may not reach the wound.

The transmission portion 218 may be constructed from any suitable material for conveying high frequency vibrational energy, such as a silicone gel, a silicone adhesive, Cica Care silicone gel, Durafiber that may be wetted out, or ultrasound connection gel surrounded by a film bubble. A series of silicone strands may be embedded in the delivery layer, thereby allowing for multiple pathways.

A wound contact layer may be positioned beneath the delivery layer. The wound contact layer may be constructed from any suitable material such as disclosed herein this section or elsewhere in the specification. For example, the wound contact layer may be constructed from polyurethane alone or polyurethane coated with a silicone adhesive on the bottom, top, or both the bottom or the top of the wound contact layer. The wound contact layer itself may be replaced with a silicone adhesive layer. The wound contact layer may be constructed as a film layer coated in an acrylic constructional adhesive.

One of skill in the art will understand that the wound contact layer and the delivery layer, outside of the transmission portion, may be porous thereby allowing wound exudate to pass through the wound contact layer and the delivery layer, to be absorbed within the absorbent layer. Negative pressure therapy and ultrasound therapy may be simultaneously applied to the wound treatment apparatuses of FIG. 2. NPWT may be applied in an alternating fashion with therapeutic ultrasound. Advantageously, application of negative pressure may serve to draw the dressing and/or substrate downward toward the wound, thereby bringing the transducers and/or transmission medium(s) directly into contact with tissue and/or exudate. Bringing the dressing or substrate into direct contact with tissue and/or exudate advantageously may reduce signal lost through transmission through air.

One of skill in the art will further understand that the positioning of the various layers and components as described in FIG. 2 is for illustrative purposes, and the transducer may be positioned in various locations within the dressing. For example, the transducer may be placed directly against the wound contact layer, on the sides of the dressing, near the center of the dressing, off-center within the dressing, or any other suitable position.

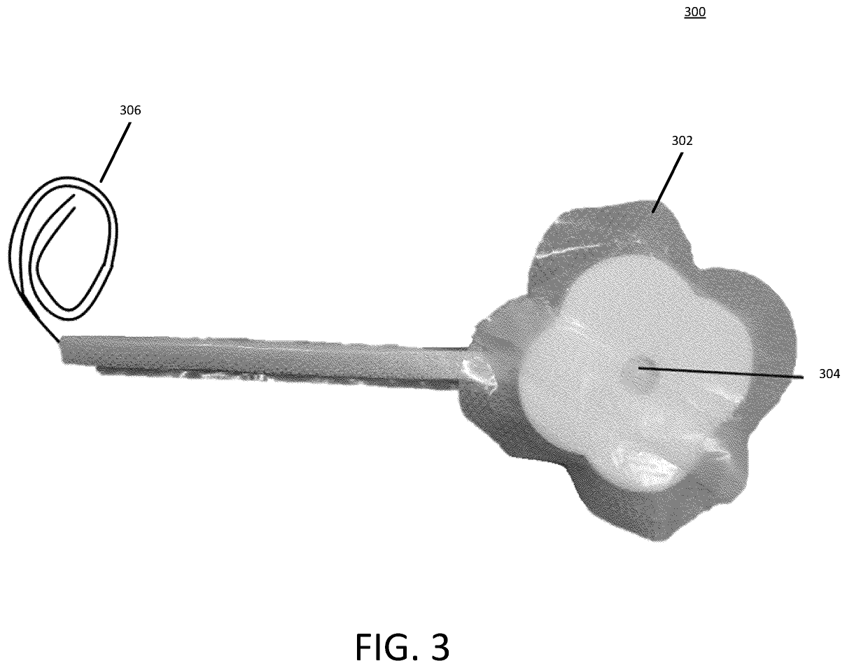

FIG. 3 depicts a bottom view of a lobed therapeutic ultrasound wound treatment apparatus 300, similar to the apparatus of FIG. 2. Here the shape of the apparatus may be in the form of a four-lobed dressing 302. The four-lobed dressing can connect to an electrical signal generator via wires 306. However, the dressing may be oval shaped, rectangular, single-lobed, double-lobed, triple-lobed, or comprise five or more lobes. The dressing may further be shaped according to any shape or size disclosed herein this section or elsewhere in the specification. Here, as in FIG. 2, the transmission portion 304 may be centrally located within the dressing portion of the apparatus.

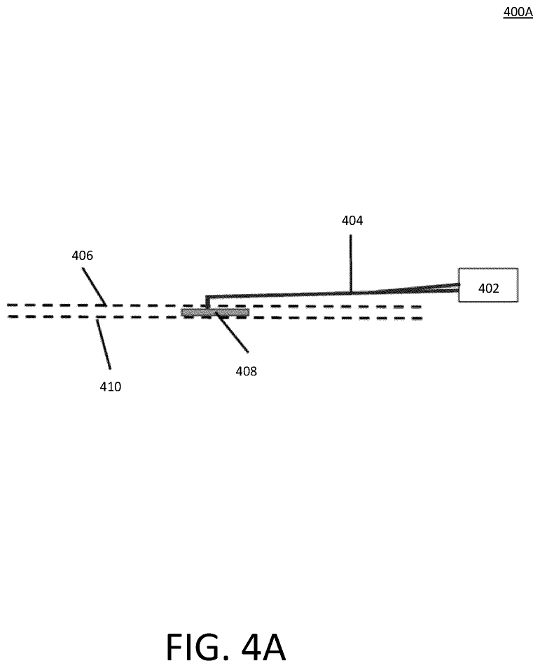

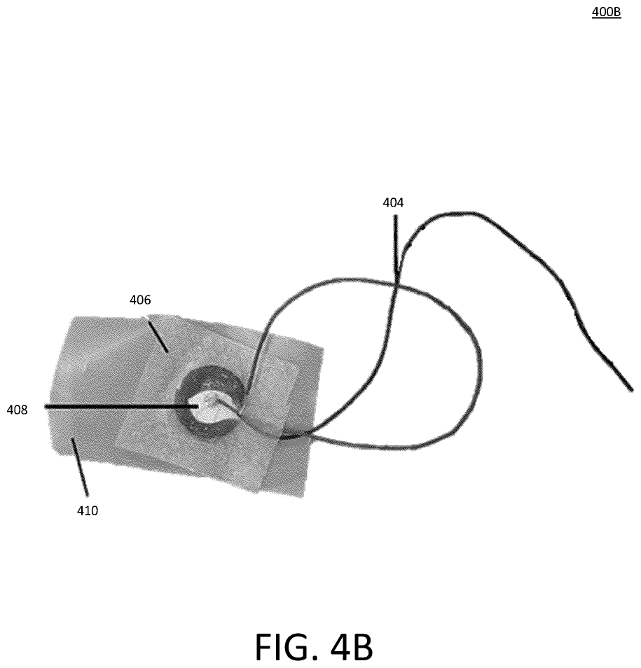

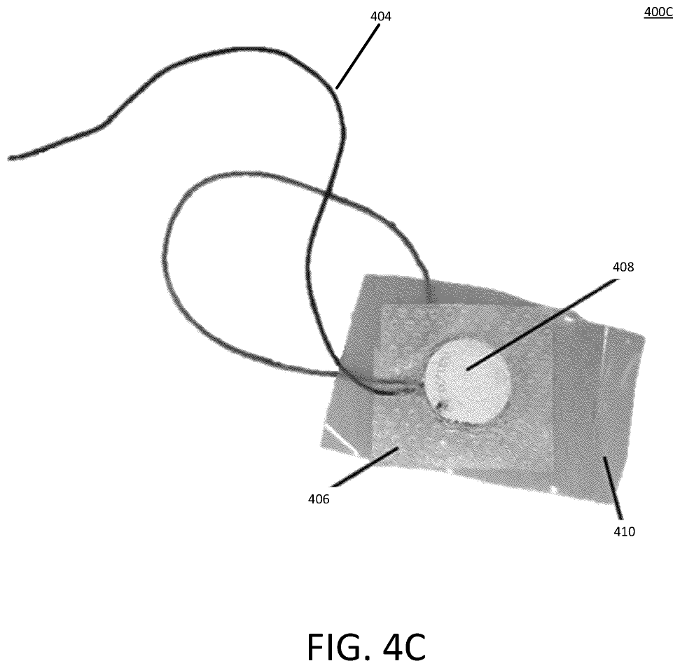

FIG. 4A depicts a therapeutic ultrasound wound treatment apparatus 400A with some similarities to the apparatuses of FIGS. 2 and 3. An electrical signal generator 402 (e.g., an ultrasonic frequency electrical signal generator) connects to a transducer 408 via wires 404. However, here the transducer 408 may be placed directly over the wound contact layer 410. The wound contact member may be any material disclosed herein, such as silicone. The transducer may then be encapsulated by cover layer 406. FIG. 4B is a view 400B of the apparatus of FIG. 4A, shown from the top, while FIG. 4C is a view 400C of the apparatus of FIG. 4A, shown from the bottom. Wires 404, cover layer 406, transducer 408, and wound contact layer 410 are shown.

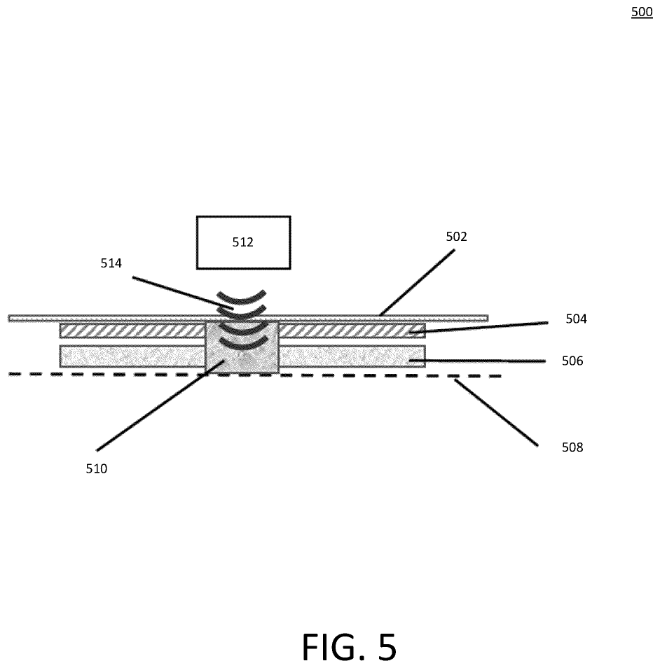

FIG. 5 depicts a wound dressing 500 that can be used with an ultrasound delivery apparatus, similar to the apparatuses of FIGS. 2 and 3. Here, the wound dressing 500 includes a cover layer 502, an absorbent layer 504, a delivery layer 506, and a wound contact layer 508. However, a transmission portion extends from the wound contact layer through the delivery layer and the absorbent layer to the cover layer 502. The positioning of the transmission portion now allows a pathway from the top of the dressing to the bottom of the dressing. Therefore, a transducer 512 may be positioned over the transmission portion 510 and deliver therapy through the dressing. The transducer may be mounted to the top of the dressing or may be an external device that can be applied and removed at will.

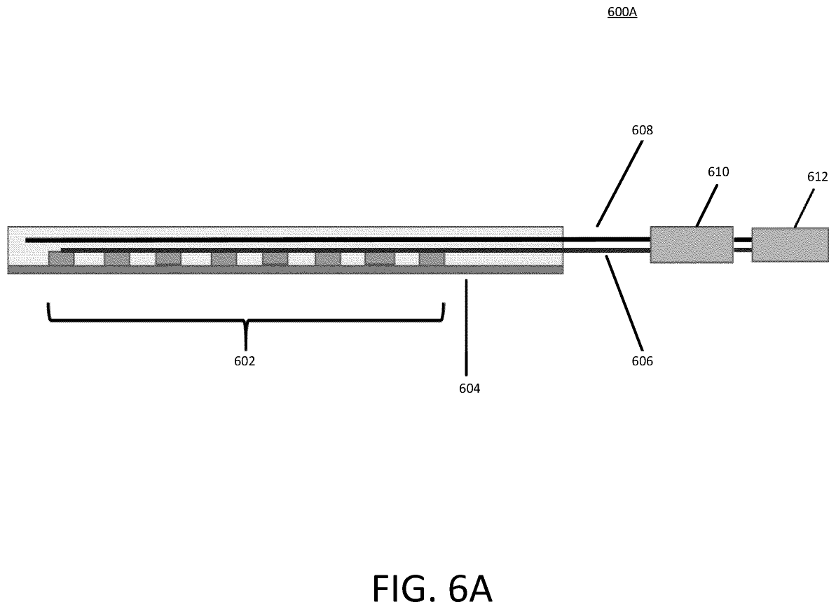

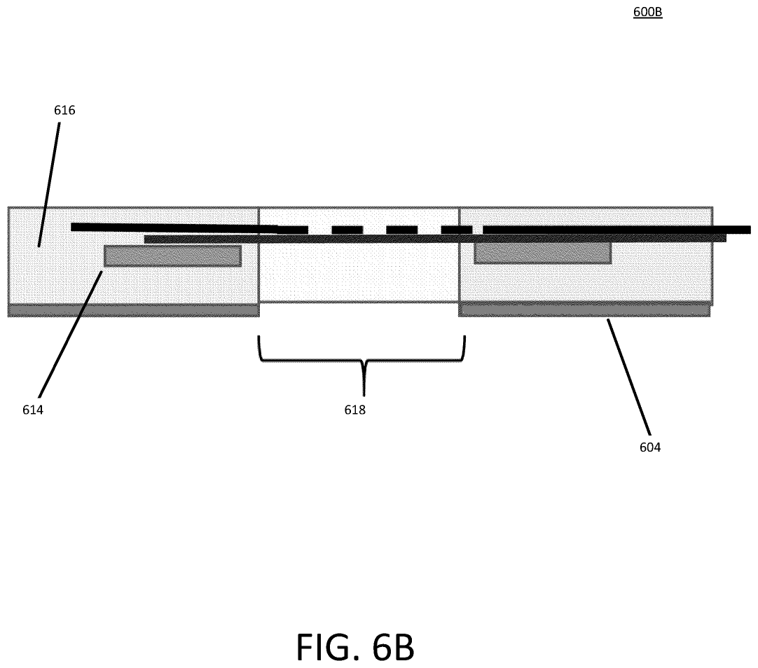

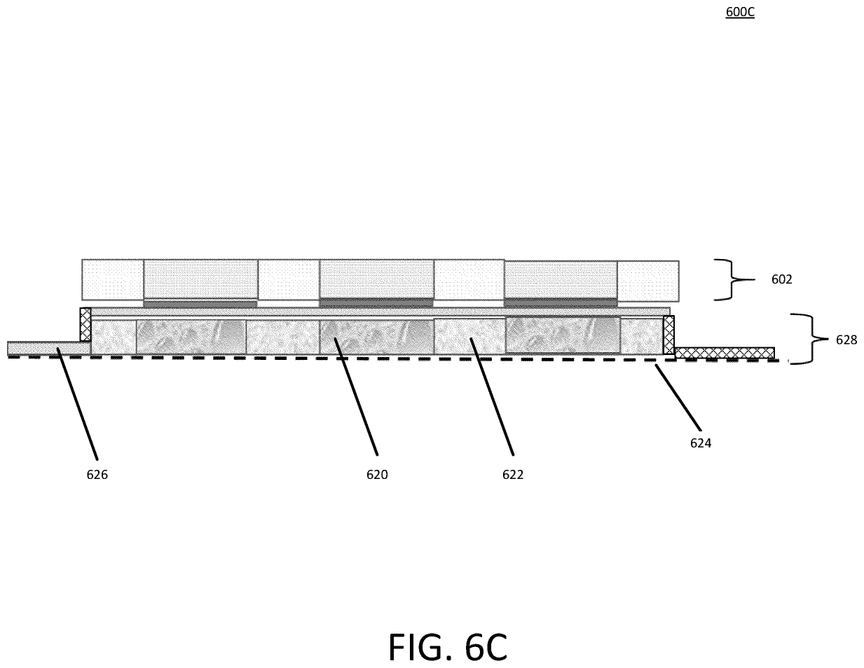

FIG. 6A depicts a therapeutic ultrasound wound treatment apparatus 600A with some similarities to the apparatuses of FIGS. 2, 3, 4A, 4B, and 4C. The apparatus 600A can include an array of transducers 602. The array of transducers 602 can be attached to a flexible printed circuited board of the apparatus 600A. The array of transducers 602 can be encapsulated in silicone with perforations between the transducers of the array and prints electrical connections. The perforations can allow for breathability. The array of transducers 602 can include one or more removable and/or reusable transducers. The array of transducers 602 can be used in multiple therapies. For example, the array of transducers 602 can be used in musculoskeletal therapies such as sports medical and orthopedic applications. The apparatus 600A can include an adhesive layer 604. The adhesive layer 604 can be adhesive coated and may enable the apparatus 600A to be attached to the surface through which a function (e.g., an ultrasound function) is to be delivered. The function can be delivered directly through the skin or through a dressing that includes a transmitting window. The function can be delivered directly through the skin where the skin is unbroken. The apparatus 600A can include a printed circuit track 606 and a cover layer 608, such as thermoplastic polyurethane (“PU”) film. The cover layer 608 can protects wounds and maintain moist environment in the wound. The cover layer 608 may be occlusive.

The apparatus 600A can include a connector 610 configured to receive a drive signal from control circuitry 612 of a therapy source. The control circuitry 612 can include or be connected to a signal generator, a driving unit, etc. The control circuitry 612 can include logic circuitry, such as one or more controllers or processors. The apparatus 600A can receive the drive signal from control circuitry 612 and, based on the drive signal, cause the therapy source to provide stimulation to the array of transducers 602. By providing the stimulation to the array of transducers 602 based on the drive signal, the apparatus 600A may cause the array of transducers 602 to perform a wound therapy function (e.g., an ultrasound function). The control circuitry 612 can be tethered to the apparatus 600A. The apparatus 600A can include the control circuitry 612. Components 602, 604, 606, 608, and 610 can collectively form a wound dressing. The apparatus 600A can include a memory in order to store, for example, pairing data and/or identification data corresponding to the connection of the control circuitry 612 and the apparatus 600A. The memory can indicate valid control circuitry 612 to be connected to the apparatus 600A. The memory can store a timer and a corresponding lifetime threshold. The timer can indicate the time of connection of the control circuitry 612 and the apparatus 600A and the lifetime threshold may indicate a time period (or operational lifetime) over which the wound dressing can be used for treating a patient prior to being discarded or refurbished for another use (such as, for treating a different patient).

Based on the stored connection data (e.g., the pairing data, the identification data, and/or the timer), the control circuitry 612 can be configured to power the array of transducers 602 such that the array of transducers 602 performs a therapy function for a limited time period. The control circuitry 612 can provide power to the array of transducers 602 via a wired electrical connection such as via the printed circuit track 606. The control circuitry 612 can also provide power to the array of transducers 602 wirelessly. For example, the control circuitry can power the array of transducers 602 by providing a wireless signal (e.g., radio waves) to the array of transducers 602. The control circuitry 612 can include a wireless transmitter (e.g., a Radio-Frequency Identification (“RFID”) transmitter) that wirelessly transmits a signal to the array of transducers 602 and powers the array of transducers 602. The control circuitry 612 can wirelessly power the array of transducers via RFID wireless power transfer, Qi wireless power transfer, inductive charging, resonant inductive coupling, power beaming, capacitive coupling, magnetodynamic coupling, microwave charging, laser beam charging, atmospheric plasma channel coupling, energy harvesting, or the like. The apparatus 600A can include a fuse with the dressing, such as in the connector 610. The fuse may allow the control circuitry 612 to disable the dressing (such as, disable the array of transducers 602) based on determining that the lifetime threshold of the apparatus 600A has been exceeded.

FIG. 6B depicts a therapeutic ultrasound wound treatment apparatus 600B with some similarities to the apparatuses of FIGS. 2, 3, 4A, 4B, 4C, and 6A. The apparatus 600B can include one or more transducers 614 encapsulated in silicone 616. Each transducer 614 may be encapsulated in a particular section of silicone 616 and may be attached to a surface via the adhesive layer 604. The apparatus 600B may include perforations 618 between the transducers 614 and the electrical circuit.

FIG. 6C depicts a therapeutic ultrasound wound treatment apparatus 600C with some similarities to the apparatuses of FIGS. 2, 3, 4A, 4B, 4C, 6A, and 6B. The apparatus 600C can include an array of transducers 602. The array of transducers 602 can be applied over a cover layer 626 (such as, top film) of a wound dressing 628. The array of transducers can perform the function through one or more transmission windows 620 (e.g., ultrasound transmission windows) in the wound dressing 628. The one or more transmission windows 620 can include one or more sections encapsulated in silicone. The wound dressing 628 can include one or more sections of absorbent material 622. The one or more sections of absorbent material 622 may be absorbent in order to absorb fluid, such as wound exudate. The wound dressing 628 can include a wound contact layer 624. Areas of the wound contact layer 624 corresponding to the one or more transmission windows 620 may be solid to increase transmission of a signal (e.g., an ultrasound signal). Areas of the wound contact layer 624 not corresponding to the one or more transmission windows 620 may be perforated to facilitate fluid transmission.

### Managing Operation of Wound Dressings

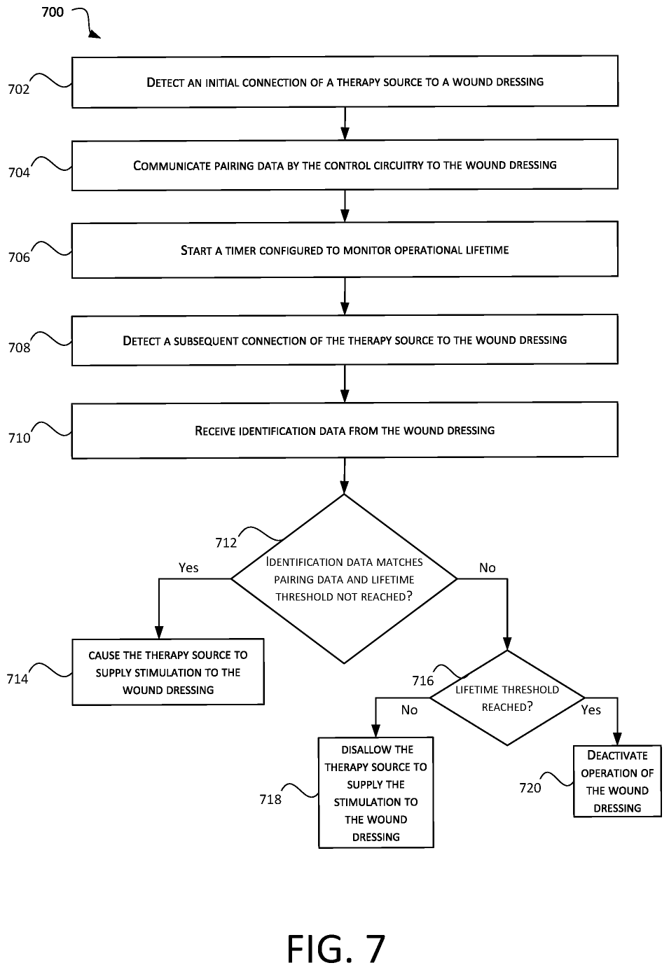

With reference to FIG. 7, an illustrative process 700 will be described for managing operation of a wound dressing, such as any of the wound dressings described herein. The process 700 may be implemented, for example, by the control circuitry of FIG. 6A.

The process 700 can begin at block 702, where the process detects an initial connection of the therapy source to the wound dressing. The therapy source can be configured to be connected to the wound dressing. Further, the therapy source can be configured to supply stimulation to the wound dressing. The connection of the therapy source to the wound dressing can correspond to a physical connection of the therapy source to the wound dressing (e.g., an electrical connection). The connection of the therapy source to the wound dressing can correspond to a wireless connection between the therapy source and the wound dressing. The the connection of the therapy source to the wound dressing can correspond to a designation that the therapy source corresponds to the wound dressing. For example, the process 700 may receive a designation that a user (e.g., caregiver) has linked the therapy source to the wound dressing. The therapy source may be linked to the wound dressing via an interaction of the user with a user interface (e.g., a graphical user interface). For example, the user may link the therapy source to the wound dressing via a website, a mobile device application, a computer application, a user interface of the therapy source and/or the wound dressing, an email, a text, or any other user interface.

A wound therapy apparatus can include the therapy source and the control circuitry that provides or cause provision of a driving signal to the therapy source to enable the therapy source to provide the stimulation to the wound dressing. A kit can include the wound therapy apparatus and one or more wound dressings (e.g., the wound dressing connected to the therapy source). The therapy source can include at least one of a source of vibrational energy, a light source, or a negative pressure source. The wound dressing can be configured to cover a wound and can include one or more electronic components. The wound dressing can further include a connector configured to connect the wound dressing to the therapy source. The connector may include a fuse positioned in the connector. The one or more electronic components can be configured to provide therapy to the wound in response to the stimulation received from the therapy source. The one or more electronic components can include at least one ultrasonic transducer that can be configured to apply vibrational energy to the wound. The control circuitry may include a driving circuit configured to provide a driving signal to the wound dressing. The control circuitry may detect the initial connection of the therapy source to the wound dressing based on an electrical connection of the therapy source to the wound dressing. For example, the control circuitry may be configured to determine that the therapy source is electrically connected to the wound dressing. The control circuitry may detect the initial connection based on one or more signals that are passed between the therapy source to the wound dressing. The control circuitry can detect the initial connection by obtaining data associated with the wound dressing. For example, the control circuitry can obtain an Internet Protocol (“IP”) address associated with the wound dressing.

The therapy source and the wound dressing may include network-equipped computing devices, for example desktop computers, laptops, smartphones, tablets, e-readers, gaming consoles, and the like. The therapy source and the wound dressing can communicate via the network to establish a connection and/or to prepare the wound dressing to perform a function. The control circuitry can further determine that the therapy source has been initially connected to the wound dressing by detecting a connection of the therapy source and the wound dressing over the network. The network can include any appropriate network, including an intranet, the Internet, a cellular network, a local area network or any other such network or combination thereof. Protocols and components for communicating via the Internet or any of the other aforementioned types of communication networks are known to those skilled in the art of computer communications and thus, need not be described in more detail herein.

In response to detecting the initial connection of the therapy source to the wound dressing, at block 704 the process 700 can communicate pairing data to the wound dressing. The pairing data may correspond to one or more unique codes associated with the control circuitry and/or the therapy source. The process 700 can communicate the pairing data based on determining that the therapy source and the wound dressing have been initially connected. Additionally or alternatively, the process 700 can be configured to communicate the pairing data based on receiving an indication that the therapy source and the wound dressing have not been previously connected (e.g., by comparing an identifier of the wound dressing with stored pairing data).

The pairing data can include a designation of the therapy source and/or the wound dressing. The pairing data can designate an exclusive relationship between the therapy source and the wound dressing. For example, the pairing data can indicate that the wound dressing is configured to function only when connected to the therapy source. The pairing data can designate a limited number of therapy sources that have been approved for a particular wound dressing and/or a limited number of wound dressings that have been approved for a particular therapy source. For example, a particular therapy source may be approved for a first wound dressing and, based on the first wound dressing being removed and replaced with a second wound dressing, the particular therapy source may also be approved for a second wound dressing. Therefore, the pairing data can ensure that the therapy source is approved for a limited number of wound dressings and/or the wound dressing is approved for a limited number of therapy sources. One or more of the control circuitry, the therapy source, and/or the wound dressing can store the pairing data in association with additional connection data and/or stimulation data. For example, the control circuitry can be configured to cause the wound dressing to store the pairing data in a memory of the wound dressing. The control circuitry and/or the wound dressing can store a plurality of pairing data corresponding to multiple paired connections. The communication of the pairing data, may cause storing of one or more unique codes corresponding to the pairing data in memory of the wound dressing. The control circuitry may generate new pairing data each time a new wound dressing is connected.

In response to detecting the initial connection of the therapy source to the wound dressing, at block 706 the process 700 can start a timer configured to monitor the operational lifetime of the wound dressing. The timer can be local to (such as, stored in memory of) one or more of the control circuitry, the wound dressing, and/or the therapy source. The process 700 can start the timer based upon the initial connection of the therapy source to the wound dressing. Additionally or alternatively, the process 700 can start the timer based on providing the stimulation to the wound dressing. For example, the process 700 can start the timer when stimulation is initially provided by the one or more electrical components of the wound dressing. Additionally or alternatively, the process 700 can start the timer based on communicating the pairing data to the wound dressing. The process 700 can, via the timer, monitor a total time period for which the one or more electrical components are providing stimulation to the wound dressing. For example, the timer may be configured to monitor a total time for which the components provide stimulation. Additionally or alternatively, the process 700 can monitor a total time for which the therapy source and the wound dressing have been connected. For example, the timer may be configured to monitor a time from the initial connection of the therapy source and the wound dressing. Additionally or alternatively, the process 700 can monitor a total time for which the therapy source and the wound dressing have been actively connected. For example, the timer may be configured to monitor a time from the connection of the therapy source and the wound dressing to disconnection. Additionally or alternatively, the process 700 can monitor a total time for which the therapy source and the wound dressing have been paired (e.g., a time since the pairing data was communicated by the control circuitry to the wound dressing). One or more of the control circuitry or the wound dressing can store the timer (such as, in memory) to monitor the operational lifetime of the wound dressing.

The therapy source and the wound dressing may subsequently be disconnected. For example, the therapy source and the wound dressing may be disconnected in order to allow access to the wound and/or the wound dressing for cleaning, showering, a medical procedure (e.g., an MRI), etc. The therapy source and the wound dressing may be disconnected for a period of time and later reconnected. Further, the therapy source and the wound dressing may be disconnected and the therapy source may be connected to a new wound dressing or the wound dressing may be connected to a new therapy source. In order to ensure that the therapy source is authorized to provide the stimulation to the connected wound dressing, the control circuitry can perform a handshake with the wound dressing. The handshakes can verify that the wound dressing corresponds to a known and authorized wound dressing.

At block 708, the process 700 can detect a subsequent connection of the therapy source to the wound dressing. The process 700 may detect the subsequent connection of the therapy source to the wound dressing based on a subsequent electrical connection of the therapy source to the wound dressing. For example, the process 700 may be configured to determine that the therapy source is electrically connected to the wound dressing after an initial electric connection of the therapy source and the wound dressing. Additionally or alternatively, the process 700 may detect the subsequent connection based on one or more signals that are passed (for instance, wirelessly) between the therapy source to the wound dressing.

In order to determine whether the therapy source is authorized to supply stimulation to the wound dressing, in response to detecting the subsequent connection of the therapy source to the wound dressing, at block 710 the process 700 can receive identification data from the wound dressing. The process 700 may perform a handshake between the wound dressing and the therapy source in order to verify that the therapy source is authorized to supply stimulation to the wound dressing. The identification data may correspond to an identifier of the particular wound dressing. For example, the identification data may be a unique identifier corresponding to the particular wound dressing. The process 700 may perform the handshake in order to verify that the identification data from the wound dressing corresponds to stored pairing data, which can indicate previous pairing between the wound dressing and the therapy source. In order to perform the handshake with the wound dressing, the process 700 may receive data from the wound dressing and/or send data to the wound dressing. For example, the process 700 may receive identification data from the wound dressing and send corresponding pairing data to the wound dressing. Based on the corresponding identification data and the pairing data, the control circuitry and/or the wound dressing may confirm that the control circuitry and the wound dressing are linked. The wound dressing may be configured to provide identification data to the control circuitry upon each subsequent connection of the wound dressing and the therapy source.

The initial connection, the subsequent connection, the transmission of pairing data, and/or the transmission of identification data may correspond to an encrypted exchange between the control circuitry and the wound dressing. The exchange between the control circuitry and the wound dressing may be wirelessly encrypted according to any one or more encryption standards. For example, the exchange may correspond to a pair of keys (e.g., a corresponding public key and private key). As part of an initial communication between the wound dressing and the control circuitry, the control circuitry may provide the corresponding public key to the wound dressing for use in encryption and/or decryption. The wound dressing may encrypt the identification data using the public key and transmit the encrypted identification data to the control circuitry. The control circuitry may receive the encrypted identification data and decrypt the encrypted identification data using the private key. As the public key and the private key are a corresponding pair of keys, the encryption and subsequent decryption of the identification data may provide further validation that the therapy source is authorized to supply stimulation to the wound dressing. Additional forms of encrypting the communications between the control circuitry and the wound dressing may be used. For example, a particular key known by both the control circuitry and the wound dressing may be used to both encrypt and decrypt the communications. Symmetric encryption and/or asymmetric encryption can be used to encrypt communications between the control circuitry and the wound dressing. For example, Data Encryption Standard (“DES”) encryption, Advanced Encryption Standard (“AES”) encryption, Rivest-Shamir-Adleman (“RSA”) encryption, Twofish encryption, Blowfish encryption, Threefish encryption, Triple DES encryption, Elliptic Curve Cryptography (“ECC”) encryption, or any other type of encryption can be used to encrypt the communications.

At block 712, the process 700 can determine whether the identification data matches the pairing data and whether a lifetime threshold has not been reached. If there is a match and the lifetime threshold has not been reached, the process 700 can transition to block 714, where the process can cause the therapy source to supply stimulation to the wound dressing. By determining that the identification data matches the pairing data, the process 700 may determine that the therapy source is authorized to provide stimulation to the wound dressing. As described herein, the lifetime threshold may correspond to a threshold period of time for which the wound dressing may be used for treating a patient. By determining the lifetime threshold has not been reached, the process 700 may determine that the wound dressing can still be used for treatment. As described herein, a timer can be maintained to track operational lifetime. The timer can be compared to the lifetime threshold. In some cases, the lifetime threshold can be indicative of a time duration over which the therapy source can be operational, and determining whether the lifetime threshold has been reached can include determining that the therapy source has not surpassed a threshold level of stimulation that can be provided to the wound dressing.

In response to determining at block 712 that the identification data does not match the pairing data or that the lifetime threshold has been reached, the process 700 can transition to block 716. At block 716, the process 700 can determine whether the lifetime threshold has been reached. If not, the process 700 can transition to block 718, where the process can disallow the therapy source to supply the stimulation to the wound dressing. The process 700 may determine that the identification data does not match the pairing data and determine that the therapy source is not authorized to provide stimulation to the particular wound dressing. This can be due to another therapy source being previously paired with the wound dressing.

If the process 700 determines at block 716 that the lifetime threshold has been reached, the process can transition to block 720. At block 720, the process 700 can deactivate operation of the wound dressing. The process 700 can track multiple lifetime thresholds. For example, a first lifetime threshold may correspond to a lifetime of a particular wound dressing and a second lifetime threshold may correspond to a lifetime of another particular wound dressing. Based upon determining that a lifetime threshold corresponding to a particular wound dressing has been reached, the process 700 can deactivate operation of that particular wound dressing. In order to deactivate operation of the wound dressing, the process 700 can be configured to perform one or more operations. For example, in order to deactivate operation of the wound dressing, the process 700 can apply an electrical signal configured to sever at least one electrical connection in the wound dressing. For instance, the electrical signal can be configured to deactivate a fuse of the wound dressing. The fuse can be positioned in the connector of the wound dressing. In another example, in order to deactivate operation of the wound dressing, the process 700 can at least partially erase a memory of the wound dressing. In yet another example, in order to deactivate operation of the wound dressing, the process 700 can store a deactivation code in the memory of the wound dressing.

The process 700 can be configured to detect an initial connection of the therapy source to another wound dressing. The another wound dressing may include another one or more electronic components configured to provide therapy to the wound or to another wound in response to the stimulation received from the therapy source. The process 700 can additionally or alternatively communicate another pairing data to the another wound dressing in response to detecting the initial connection. The process 700 can start another timer in response to detecting the initial connection. The another timer can be configured to monitor the operational lifetime of the another wound dressing. The process 700 can detect a subsequent connection of the therapy source to the wound dressing or to the another wound dressing. In response to detecting the subsequent connection, the process can request and receive from the wound dressing or the another wound dressing, the identification data. In response to determining that the identification data matches the pairing data and that the lifetime threshold has not been reached, the process 700 can cause the therapy source to supply the stimulation to the another wound dressing. In response to determining that the identification data matches the another pairing data and that the another lifetime threshold has not been reached, the control circuitry can cause the therapy source to supply the stimulation the another wound dressing. The process 700 can be configured to cause the therapy source to provide different levels of stimulation (e.g., different voltages, currents, powers) to the different wound dressings. For example, the level of stimulation may be based on the particular wound dressing. Additionally or alternatively, the process 700 can disallow the therapy source to supply the stimulation to at least one of the wound dressing or the another wound dressing in response to determining at least one of: the identification data does not match the pairing data, the identification data does not match the another pairing data, the identification data matches the pairing data and the lifetime threshold has been reached, or the identification data matches the another pairing data and the another lifetime threshold has been reached. The process 700 can deactivate operation of the another wound dressing in response to determining that the another lifetime threshold has been reached. The lifetime threshold and the another lifetime threshold can be the same lifetime threshold or a different lifetime threshold. For example, the lifetime threshold may be a general lifetime threshold or may be specific to a wound dressing or a group of wound dressings. One or more of the control circuitry and/or the another wound dressing can store the another pairing data in association with additional connection data and/or stimulation data. For example, the control circuitry can be configured to cause the another wound dressing to store the another pairing data in a memory of the another wound dressing.

In some cases, verification that therapy is being applied to the wound can be made. Verification that therapy is being applied can be used to start the timer configured to monitor the operational lifetime of the wound dressing. The verification can be made by the control circuitry and/or the wound dressing. For example, one or more temperature sensors can measure the temperature of one or more ultrasonic transducers and/or tissue of a patient. The measured temperature can be used to verify that vibrational energy is being applied to the patient. For instance, temperature of the one or more ultrasonic transducers can increase when energy is being applied to the one or more ultrasonic transducers to cause application of vibrational energy. As another example, temperature of tissue of the patient under the wound dressing and/or surrounding the wound dressing can increase due to the application of vibrational energy. Temperature measured by the one or more sensors can be compared to one or more temperature thresholds to detect the temperature increase. Additionally or alternatively, feedback electrical current can be measured in one or more ultrasonic transducers. Responsive to determining that the feedback electrical current satisfies a current threshold, a determination can be made that vibrational energy is being applied to the patient. Additionally or alternatively, impedance (or conductance) of the tissue of tissue of the patient under the wound dressing and/or surrounding the wound dressing can vary due to the application of vibrational energy. For instance, in some cases, compression of the tissue can cause decrease in the impedance, and expansion of the tissue can cause increase in the impedance. One or more impedance sensors can be used to measure the impedance variation. Measured impedance variation can be compared to one or more impedance thresholds to determine whether vibrational energy is being applied to the tissue of the patient. Measured impedance variation can be processed to determine whether it satisfies a variation pattern indicative of the application of vibrational energy.

Any of the wound dressings disclosed herein can support one or more temperatures sensors, impedance sensors, or the like used for the verification. In some cases, any of the sensor integrated dressings supporting one or more temperatures sensors, impedance sensors, or the like can be used. Examples of such sensor integrated dressings are disclosed in U.S. Patent Publication No. 2019/0290496 and/or International Patent Application No. PCT/EP2020/052111, each of which is incorporated by reference in its entirety.

Any of the verification approaches described herein (such as, temperature measurement, feedback current measurement, or impedance measurement) can be used to detect whether any one or more of the ultrasonic transducers is not working properly. This can be due to malfunction of an ultrasonic transducer, poor power supply, or the like. Indication that any one or more of the ultrasonic transducers is not working properly can be provided to a user.

In some cases, verification that therapy is being applied to the wound can include detection of a change in pressure. When negative pressure is being applied (such as, along with the application of vibrational energy), reduction of pressure can be detected and used for the verification. One or more of a pressure sensor, pressure switch, strain gauge, or the like can be used to detect change of pressure at the wound (such as, under a wound dressing, in a fluid flow path connecting the wound to the source of negative pressure, or the like). Approaches for detecting the change of pressure are described in International Patent Application No. PCT/EP2020/072663, filed on Aug. 18, 2020, which in incorporated by reference in its entirety. The timer configured to monitor the operational lifetime of the wound dressing can be started responsive to a detection of a pressure drop (or reduction of pressure) that satisfies a pressure threshold. The detection can be performed over a threshold duration of time.

### Other Variations

Although negative pressure wound therapy is described, the systems, devices, and/or methods disclosed herein can be applied to other types of therapies usable standalone or in addition to TNP therapy. Systems, devices, and/or methods disclosed herein can be extended to any medical device, and in particular any treatment or monitoring device. For example, systems, devices, and/or methods disclosed herein can be used with devices that provide one or more of ultrasound therapy, oxygen therapy, light therapy, neurostimulation, microwave therapy, active agents, antibiotics, antimicrobials, or the like. Such devices can in addition provide TNP therapy. The systems and methods disclosed herein are not limited to medical devices and can be utilized by any electronic device.

While certain embodiments have been described in the context of managing operation of wound dressings (such as, limited use wound dressings), the devices, and/or methods disclosed herein can be used to manage operation of treatment devices (such as, limited use devices). For example, the Versajet debridement system sold by Smith & Nephew includes a handpiece configured to be connected to a console that controls the operation of the handpiece. The approaches disclosed herein can be used to manage the operation of the handpiece, which can be a limited use device.

While certain embodiments have been described in the context of treating a wound, the devices, and/or methods disclosed herein can be applied to wound monitoring. For example, a wound dressing supporting one or more sensors can be positioned on a wound and collect data from the wound. The wound dressing can communicate (over wired or wireless connection) with an external control device. For example, such wound monitoring system is disclosed in U.S. Patent Application Publication No. 2019/0290496. The approaches disclosed herein can be used to manage the operation of wound dressings configured to monitor wounds (any of which can be a limited use dressing). A wound dressing configured to monitor a wound can be paired with a control device, which can communicate with the dressing but not supply stimulation or therapy to the dressing. Otherwise, managing the operation of the wound dressing configured to monitor a wound can be performed using any of the approaches described herein.

Any of transmission of data described herein can be performed securely. For example, one or more of encryption, https protocol, secure VPN connection, error checking, confirmation of delivery, or the like can be utilized.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added. For instance, the various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

User interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, displayed in any suitable order.

Conditional language used herein, such as, among others, “can,” “could”, “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

1. An apparatus for treating a wound, the apparatus comprising:
a treatment source configured to supply stimulation and be connected to a wound dressing covering a wound, the wound dressing comprising one or more electronic components configured to provide treatment to the wound in response to the stimulation received from the treatment source; and
a control circuitry configured to:
in response to detecting an initial connection of the treatment source to the wound dressing:
communicate pairing data to the wound dressing; and
start a timer configured to monitor operational lifetime of the wound dressing;
in response to detecting a subsequent connection of the treatment source to the wound dressing:
receive identification data from the wound dressing;
in response to a determination that the identification data matches the pairing data and that the timer has not reached a lifetime threshold, cause the treatment source to supply the stimulation to the wound dressing; and
in response to a determination that the identification data does not match the pairing data or that the timer has reached the lifetime threshold, disallow the treatment source to supply the stimulation to the wound dressing; and
deactivate operation of the wound dressing in response to a determination that the timer has reached the lifetime threshold.

2. The apparatus of claim 1, wherein the treatment source comprises at least one of a source of vibrational energy, a light source, or a negative pressure source.

3. The apparatus of claim 1, wherein the control circuitry is configured to deactivate operation of the wound dressing by application of an electrical signal configured to sever at least one electrical connection in the wound dressing.

4. The apparatus of claim 3, wherein the electrical signal is configured to deactivate a fuse of the wound dressing.

5. The apparatus of claim 1, wherein the control circuitry is configured to deactivate operation of the wound dressing by at least partially erasing a memory of the wound dressing.

6. The apparatus of claim 1, wherein the control circuitry is configured to deactivate operation of the wound dressing by storing a deactivation code in a memory of the wound dressing.

7. The apparatus of claim 1, wherein the control circuitry is configured to cause the wound dressing to store the pairing data in a memory of the wound dressing.

8. The apparatus of claim 1, wherein the control circuitry is configured to start the timer configured to monitor operational lifetime of the wound dressing responsive to a verification of at least one of: that treatment to the wound is being provided by the one or more electronic components or that application of negative pressure to the wound has been detected.

9. The apparatus of claim 8, wherein the verification that the treatment to the wound is being provided by the one or more electronic components is based on at least one of: detecting a temperature increase of at least one of the one or more electronic components, detecting a temperature increase of tissue in at least a portion of the wound, detecting feedback electrical current in at least one of the one or more electronic components, or detecting variation in impedance of wound tissue.

10. The apparatus of claim 1, wherein the control circuitry is further configured to:
in response to detecting an initial connection of the treatment source to another wound dressing comprising another one or more electronic components configured to provide treatment to the wound or to another wound in response to the stimulation received from the treatment source:
communicate another pairing data to the another wound dressing; and
start another timer configured to monitor operational lifetime of the another wound dressing;
in response to detecting a subsequent connection of the treatment source to the wound dressing or to the another wound dressing:
request and receive from the wound dressing or the another wound dressing the identification data;
in response to the determination that the identification data matches the pairing data and that the timer has not reached the lifetime threshold, cause the treatment source to supply the stimulation to the wound dressing;
in response to a determination that the identification data matches the another pairing data and that the another timer has not reached another lifetime threshold, cause the treatment source to supply the stimulation to the another wound dressing; and
disallow the treatment source to supply the stimulation to at least one of the wound dressing or the another wound dressing in response to a determination of at least one of: that the identification data does not match the pairing data or the another pairing data, that the identification data matches the pairing data and that the timer has reached the lifetime threshold, or that the identification data matches the another pairing data and that the another timer has reached the another lifetime threshold; and
deactivate operation of the another wound dressing in response to a determination that the another timer has reached the another lifetime threshold.

11. The apparatus of claim 10, wherein the lifetime threshold and the another lifetime threshold are the same.

12. The apparatus of claim 10, wherein the control circuitry is further configured to cause the another wound dressing to store the another pairing data.

13. (canceled)

14. (canceled)

15. (canceled)

16. A method of operating an apparatus for treating a wound, the method comprising:
by a control circuitry of the apparatus:
in response to detecting an initial connection of a treatment source of the apparatus to a wound dressing covering a wound, the treatment source configured to supply stimulation that causes the wound dressing to provide treatment to the wound:
communicating pairing data to a wound dressing; and
starting a timer configured to monitor operational lifetime of the wound dressing;
in response to detecting a subsequent connection of the treatment source to the wound dressing:
receiving identification data from the wound dressing;
in response to determining that the identification data matches the pairing data and that the timer has not reached a lifetime threshold, causing the treatment source to supply the stimulation to the wound dressing; and
in response to determining that the identification data does not match the pairing data or that the timer has reached the lifetime threshold, disallowing the treatment source to supply the stimulation to the wound dressing; and
deactivating operation of the wound dressing in response to determining that the timer has reached the lifetime threshold.

17. The method of claim 16, wherein the treatment source comprises at least one of a source of vibrational energy, a light source, or a negative pressure source.

18. The method of claim 16, further comprising, by the control circuitry, deactivating operation of the wound dressing by application of an electrical signal configured to sever at least one electrical connection in the wound dressing.

19. The method of claim 18, wherein the electrical signal is configured to deactivate a fuse of the wound dressing.

20. The method of claim 16, further comprising, by the control circuitry, deactivating operation of the wound dressing by at least one of: 1) at least partially erasing a memory of the wound dressing or 2) storing a deactivation code in the memory of the wound dressing.

21. The method of claim 16, further comprising, by the control circuitry, causing the wound dressing to store the pairing data in a memory of the wound dressing.

22. The method of claim 16, further comprising, by the control circuitry:
in response to detecting an initial connection of the treatment source to another wound dressing configured to provide treatment to the wound or to another wound in response to the stimulation received from the treatment source:
communicating another pairing data to the another wound dressing; and
starting another timer configured to monitor operational lifetime of the another wound dressing;
in response to detecting a subsequent connection of the treatment source to the wound dressing or to the another wound dressing:
requesting and receiving from the wound dressing or the another wound dressing the identification data;
in response to determining that the identification data matches the pairing data and that the timer has not reached the lifetime threshold, causing the treatment source to supply the stimulation to the wound dressing;
in response to determining that the identification data matches the another pairing data and that the another timer has not reached another lifetime threshold, causing the treatment source to supply the stimulation to the another wound dressing; and
disallowing the treatment source to supply the stimulation to at least one of the wound dressing or the another wound dressing in response to determining at least one of: that the identification data does not match the pairing data or the another pairing data, that the identification data matches the pairing data and that the timer has reached the lifetime threshold, or that the identification data matches the another pairing data and that the another timer has reached the another lifetime threshold; and
deactivating operation of the another wound dressing in response to determining that the another timer has reached the another lifetime threshold.

23. The method of claim 22, wherein the lifetime threshold and the another lifetime threshold are the same, and wherein the method further comprises, by the control circuitry, causing the another wound dressing to store the another pairing data.

24. (canceled)
